(19) United States
(12) Patent Application Publication
(10) Pub. No.: US 2022/0079641 A1
(43) Pub. Date: Mar. 17, 2022

(54) BIAXIAL CORE FASTENER

(71) Applicant: James K. Brannon, Leawood, KS (US)

(72) Inventor: James K. Brannon, Leawood, KS (US)

(21) Appl. No.: 17/478,229

(22) Filed: Sep. 17, 2021

Related U.S. Application Data
(60) Provisional application No. 63/079,581, filed on Sep. 17, 2020.

(51) Int. Cl. A61B 17/86

(52) U.S. Cl. CPC A61B 17/863; A61B 17/8685

(57) ABSTRACT

An improved biaxial core fastener for providing axial support is provided with a cylindrical housing presenting a tubular passage therethrough; an anchoring plate extending angularly from the cylindrical housing; the tubular passage having a threaded receiver; a side port extending along the cylindrical housing; a moveable axial support presenting a shaped head which is shaped with a radial passage extending therefrom the radial passage being in fluidic communication with the side port; and the moveable axial support configured for axial movement of said shaped head.

### FIELD OF THE INVENTION

The present invention is broadly directed to bone anchoring implants and, more particularly, to a biaxial core fastener which utilizes a plurality of fasteners, a fastener receiver and a centrally rotatable cylindrical head.

### BACKGROUND OF THE INVENTION

The present invention relates to an improved bone fixation device. Fixing and securing broken and injured bones have utilized a wide variety of various plates and screws for support and fixation of the bone fracture. Various bones within the body can be injured or broken. Fixation of the bones requires a variety of different sizes and shapes of plates and screws. In addition, various types of fractures also require different sized, shapes and types of fixation devices. Many of these devices do not provide sufficient variety or have sufficient attachment anchors to properly secure the injured or damaged bone. Some additional attempts at providing a bone fixation device include the use of plastic or synthetic material devices which lack sufficient structural rigidity to properly fix the healing bone. Therefore, there is a need for an improved bone fixation device which provides for sufficient attachments and sufficient structural rigidity for healing the injured or broken bone.

In some cases, treatment of damaged or injured bones involves removing or debriding damaged or necrotic bone. After removing the necrotic bone, a bone growth material is sometimes applied to help the bone heal. To remove the damaged bone and apply the growth material, specialized tools may require multiple entries into the damaged bone. It would be beneficial to provide a bone fixation device through a central portal which can be used for removal of damaged tissue, application of a growth material and secure the injured bone.

Some prior art bone fixation devices involve the use of anchors and plates to secure a broken or damaged joint. Some of these anchors involve the use of screws which are anchored into the bone tissue using walled type structures which prevent fluid from passing from or to the damaged bone area. When used to secure the damaged bone, the anchors are inserted into the bone tissue which may lead to further damage to the already damaged bone. In addition, preventing fluid from flowing to the damaged bone can lead to further decay or damage to the bone. In addition, if the damaged bone is too damaged, use of additional anchors to penetrate the already damaged bone may lead to further damage. Applying a central supporting force to the damaged bone along with the application of bone tissue has been shown to be beneficial to the treatment of damaged bone tissue. Therefore, there is a need for an improved biaxial core fastener tool which at least partially addresses some of the problems described above which includes a central single portal which can be used in a low-pressure environment for fixing the damaged bone area and which at least partially allows for passage of fluids in and from the damaged bone area.

### SUMMARY OF THE INVENTION

Certain embodiments of the present invention relate to an improved biaxial core fastener with a cylindrical housing presenting a tubular passage therethrough, an anchoring plate extending angularly from said cylindrical housing; said tubular passage presenting a threaded receiver; a side port extending along the cylindrical housing; a moveable axial support presenting a shaped head; said shaped head further comprising a radial passage extending therefrom; said radial passage being in fluidic communication with said side port; and said moveable axial support configured for axial movement of said shaped head. In another embodiment, the present inventions relates to an improved biaxial core fastener comprising a cylindrical housing configured for axial movement of an axial support; a side port positioned along said cylindrical housing; and said axial support presenting a shaped head comprising a radial passage extending radially therefrom whereby said radial passage is in fluidic communication with said side port.

Various objects and advantages of the present invention will become apparent from the following description taken in conjunction with the accompanying drawings wherein are set forth, by way of illustration and example, certain embodiments of this invention. The drawings submitted herewith constitute a part of this specification, include exemplary embodiments of the present invention, and illustrate various objects and features thereof.

### BRIEF DESCRIPTION OF THE DRAWINGS

FIG. 1 is a side perspective of an embodiment of the biaxial core fastener tool.

FIG. 2 is partially exploded, rear perspective of the embodiment of FIG. 1 with the spacer expanded rearwardly.

FIG. 3 is a top perspective, partial view of the rotatable support from FIG. 1.

FIG. 4 is a side elevation of the biaxial core fastener tool associated with an exemplary bone.

FIG. 5 is a side elevation of the biaxial core fastener tool of FIG. 1 within an exemplary bone with the central support retracted rearwardly.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail an embodiment of the biaxial core fastener tool is illustrated, generally referred to herein by reference numeral 10 formed in accordance with a first embodiment of the invention.

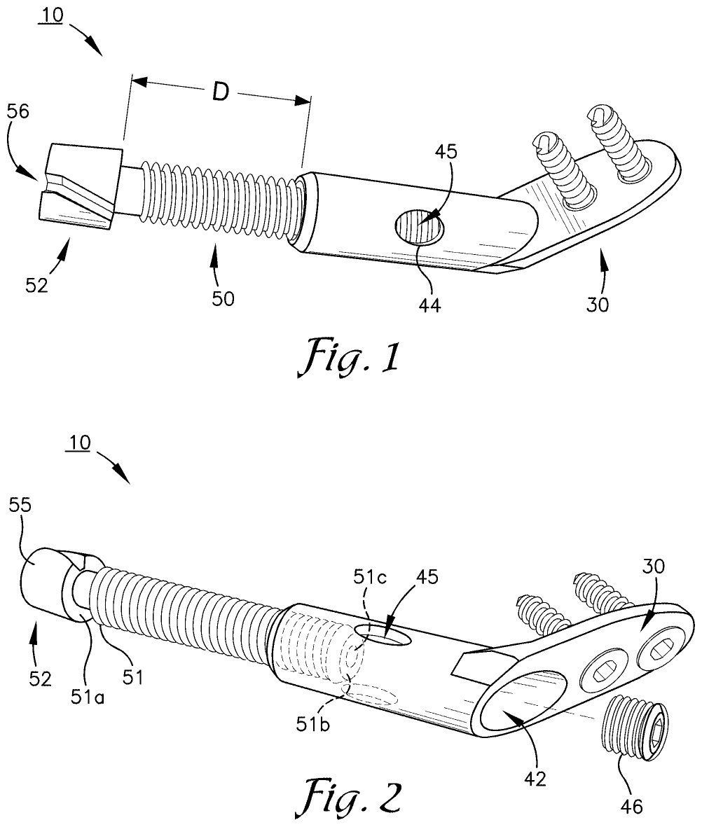

As depicted in FIGS. 1-3, an embodiment of the biaxial core fastener 10 is depicted. The biaxial core fastener 10 includes a cylindrical housing 40 extending angularly from an anchoring plate 30 and presenting a moveable axial support 50 extending through a tubular passage 42 associated with the cylindrical housing 40. Generally, the tubular passage 42 extends through the cylindrical housing 40 and includes a threaded receiver (not shown) extending circumferentially along the interior of the cylindrical housing 40 for rotation of the movable axial support 50.

The anchoring plate 30 includes a tapered end with may include a partially curved or concave surface which includes a pair of anchor fasteners for receipt of a plurality of anchors which can be threaded through the anchoring plate 30 into the desired bone structure. The anchoring plate 30 is angled from the cylindrical housing 40 to allow for angular insertion of the axial support 50.

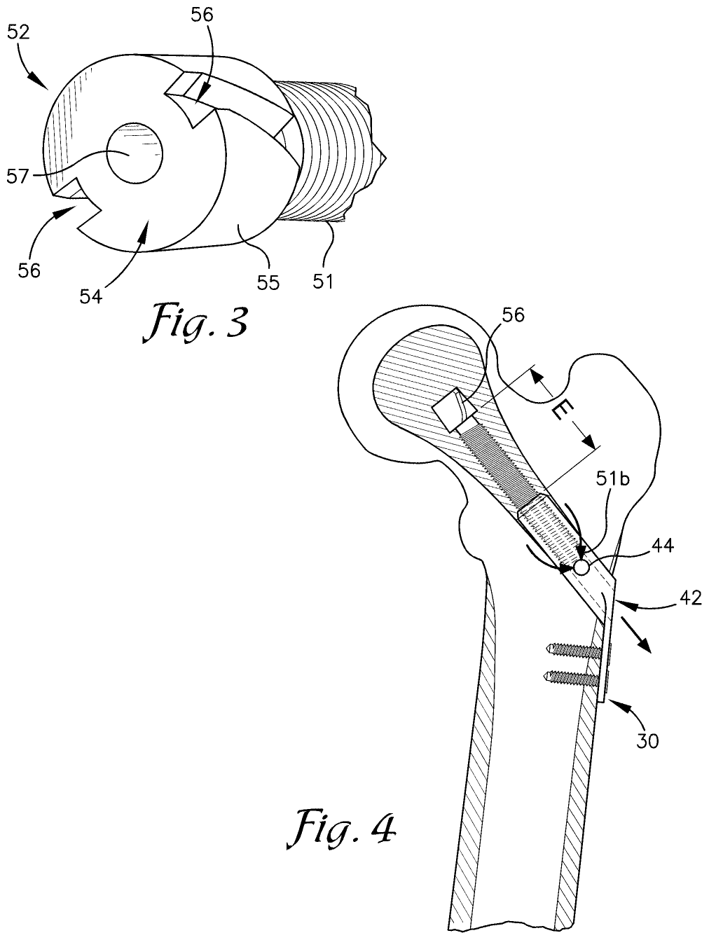

The movable axial support 50 is configured for axial movement from an outer bone surface towards an area of damaged bone to provide compressive support along the desired area of the bone. For operational movement, the moveable axial support 50 includes a shaped head 52 which as illustrated in FIG. 3. Generally, the shaped head 52 has a generally smooth upper surface with a pair of grooves spaced on opposite sides of the shaped head 52. The embodiment of the shaped head depicted in FIG. 3 is a generally cylindrical head with a smooth upper surface having a curved central depression. The shaped head 52 extends upwardly from the central depression presenting a raised radial surface with a pair of rearwardly extending channels spaced on opposite sides of the shaped head 52. Alternatively, the shaped head 52 may be substantially planar, conical or fustro-conically shaped.

The shaped head 52 is configured for operational engagement with a threaded rod 51 with corresponding threaded structures for operational receipt by the tubular passage 42. As the threaded rod 51, also referred to as a threaded member, is rotated the threaded structure moves the moveable axial support 50 along the tubular passage 42 moving the shaped head 52 towards or away from the damaged bone area.

In operation, the moveable axial support 50 generally includes axial extension of the threaded rod 51 towards the shaped head 52. As depicted in FIG. 3, the shaped head 52 has a generally concave face 54 which extends from a cylindrical sidewall 55 and includes at least one radial passage 56 which extends angularly along the cylindrical sidewall 55 from the rearwardly extending channels presenting along the concave face. The concave face 54 includes a central aperture 57 located within the central depression that extends rearwardly from the concave face 54 towards the threaded rod 51 for centrally securing the shaped head 52 to the threaded rod 51. Generally, the shaped head 52 is configured for rotation during axial movement as the shaped head 52 advances from the cylindrical housing 40 during rotation of the threaded rod 51.

At least one side port 44 is located along the cylindrical housing 40. In one embodiment of FIG. 1-2, a pair of side ports 44 extend though the sidewall of the tubular passage 42. Generally, the port 44 is in fluidic communication with the radial passage 56 associated with the shaped head 52. In one embodiment a second side port is positioned along the cylindrical housing opposite the first port 44, where both the first and second ports are in fluidic communication with the radial passage(s) 56 during for example axial movement of said axial support 50. When the moveable axial support 50 is advanced, the concave face 54 is configured for engagement with a damaged boney region, for example. When a vacuum source is supplied, the amount of fluid flowing through the radial passages 56 corresponds to the volume of fluid flowing through the side portal 44. Supplying a vacuum source at the rear of the cylindrical housing will cause fluid to flow from a surgical site of interest. As the fluid evacuates, the fluid may pass over the shaped head 52 through the radial passages 56 for transmission of the fluidic through the side port 44. As previously described, placement of the spacer 46 along the window 45 allows for adjustment of the fluid flow through the side portal as desired.

The spacer 46 is illustrated in FIG. 2. The spacer 46 is generally cylindrically shaped and has an outer diameter configured for rotational receipt along the tubular passage 42 and includes a threaded structure which is configured for rotational receipt, at the rear of the tubular passage 42. In operation, the spacer 46 is threaded into the tubular passage 42 a distance behind the moveable axial support 50. After positioning the moveable axial support 50 a distance E from the cylindrical housing 50, the spacer 46 is secured within the tubular passage 42. Generally, the spacer prevents undesired rotation of the moveable axial support 50. Generally, the spacer 46 limits undesired rotation of the moveable axial support 50. Rotation of the spacer 46 allows for axial movement along the tubular passage 42. As further depicted in FIG. 5, an embodiment of the spacer 46 operates as a closure for closing a window 45 associated with the side portal 44. As the spacer 46 is rotatable adjusted within the tubular passage 42, the spacer 46 moves laterally whereby the window 45 is more open or more closed depending on the desired volumetric flow of fluid through the side portal 44. In this way, the spacer also allows for volumetric adjustment of the fluid flowing through the side port 44. In addition, decreasing or increasing the portal opening allows for controlled flow of fluid or debris, such as but not limited to boney growth, through the side port 44.

Generally, the moveable axial support 50 includes threaded member 51 which extends from a distal to a proximate end 51a, 51b. The distal end 51a is association with the shaped head 52. The proximal end 51a is spaced opposite the distal end 51b and presents the shaped head 52 while the proximate end 51b is associated with a shaped opening 51c. The shaped opening 51c generally allows for rotation of the moveable axial support 50 through engagement of the shaped opening 51c, with for example, a rotational driver (not shown). During operation, the moveable axial support 50 is advanced axially towards the desired positioned by rotating the shaped opening 51c about the moveable axial support 50. Upon axial advancement of the moveable axial support 50 to the desired position, the shaped head 52 provides support for any received load.

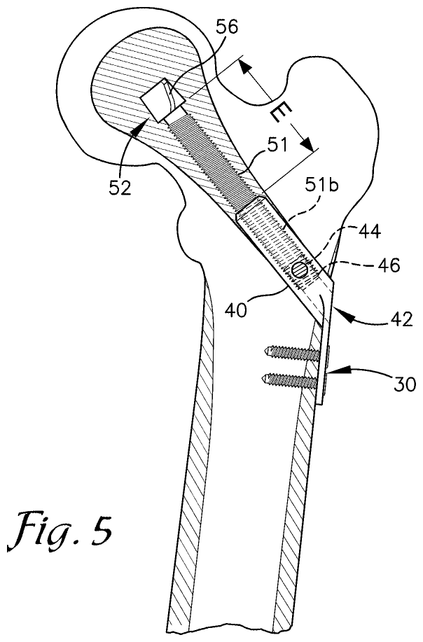

As further illustrated in FIG. 5, upon advancement of the shaped head 52 for receipt of the received load, the spacer 46 can then be inserted though the tubular passage 42 for maintaining the moveable axial support 50 in the desired position. In FIG. 5, E is slightly greater than E in FIG. 4. In FIG. 4, the moveable axial support 50 has been slightly retracted through, for example, counter-rotation of the shaped opening 51c. In the supporting position illustrated in FIG. 5, the shaped head 52 is advanced to a supporting position. As the shaped head 52 is retracted rearwardly, the fluid flow through the side port 4 is reduced. Once the shaped head 52 is positioned as desired, the fluid flow through the side port 44 may be further reduced upon placement of the spacer 46.

Generally, the radial passages 56 are in fluid communication with the side port 44 such that they attempt to equalize their pressure equilibrium. By applying a negative pressure such as an aspirator, suction or vacuum source to the proximate end of the tubular passage 42, the pressure around the side port 44 decreases causing the fluid to travel to the side port 44 from the region surrounding the debrided bone surface, behind the shaped head 52, through the radial passages 56. In this way, the user can remove various fluids from a region otherwise inaccessible based upon the engagement of the supported load. Alternatively, compressing any bone placed in the area of the removed damaged bone with the shaped head 52 will induce fluid to flow along the moveable axial support 50 and enter the side portal 44.

It is to be understood that while certain forms of the present invention have been illustrated and described herein, it is not to be limited to the specific forms or arrangement of parts described and shown.

What is claimed and desired to be secured by Letters Patent:

1. An improved biaxial core fastener comprising:
a cylindrical housing presenting a tubular passage therethrough;
an anchoring plate extending angularly from said cylindrical housing;
said tubular passage presenting a threaded receiver;
a side port extending along the cylindrical housing;
a moveable axial support presenting a shaped head;
said shaped head further comprising a radial passage extending therefrom;
said radial passage being in fluidic communication with said side port; and
said moveable axial support configured for axial movement of said shaped head.

2. An improved biaxial core fastener comprising:
a cylindrical housing configured for axial movement of an axial support;
a side port positioned along said cylindrical housing; and
said axial support presenting a shaped head comprising a radial passage extending radially therefrom whereby said radial passage is in fluidic communication with said side port.

3. The improved biaxial core fastener of claim 2 wherein said cylindrical housing further comprises an anchoring plate extending angularly from said cylindrical housing.

4. The improved biaxial core fastener of claim 2 wherein said axial support further comprises a threaded member configured for threaded receipt by said cylindrical housing.

5. The improved biaxial core fastener of claim 2 wherein said shaped head includes a concave face with a centrally located aperture extending therethrough.

6. The improved biaxial core fastener of claim 2 further comprising a second side port spaced on said cylindrical housing, opposite said side port said second side port in fluidic communication with said radial passage during axial movement of said axial support.

7. The improved biaxial core fastener of claim 2 wherein said shaped head is configured for engagement with a damaged boney region.
